(19) United States
(12) Patent Application Publication
(10) Pub. No.: US 2006/0003324 A1
(43) Pub. Date: Jan. 5, 2006

(54) Method for producing gene libraries

(76) Inventor: Volker Sieber, Wolfersdorf (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/468,391

(22) PCT Filed: Feb. 11, 2002
(86) PCT No.: PCT/EP02/01418

§ 371 Date: Mar. 12, 2004

(30) Foreign Application Priority Data
Feb. 28, 2001 (DE) 101 09 517.1

(51) Int. Cl. C12Q 1/68; C12P 19/34

(52) U.S. Cl. 435/6; 435/91.2

(57) ABSTRACT

The presented invention refers to a method for the creation of gene libraries wherein a defined number of adjacent nucleotides is exchanged and gene libraries are produced which code for protein variants having more manifold amino acid exchanges and a more homogenous distribution of mutations than can be obtained using conventional methods. DNA-strands are incorporated at random positions into a gene of interest. Then parts of the donor strands and parts of the gene sequence that is flanking these strands are removed, however, a defined number (e.g. 3) of nucleotides that originate from the donor strand remain in the gene at the place of a defined number (e.g. 3) of nucleotides of the original gene having been removed from it. Combined with a selection step after the incorporation of the donor strand into the gene it can be ensured that the nucleotides to be exchanged/introduced are in a specific reading frame. When the nucleotides of the donor strand that remain in the genes are degenerate, gene libraries can be produced with variants that have any codon at any position.

### FIELD OF INVENTION

The presented invention refers to a method for the creation of sequence variation in DNA. Especially, it can be used for the creation of libraries of genes of which the genes encode protein sequences and of which the genes in each library are distinguished to each other by the mutation (exchange) of complete codons. By using methods of selection or screening, gene variants can be isolated from these libraries that code for proteins with special properties, like for example, a change in activity or an increase in stability.

### BACKGROUND OF THE INVENTION

Enzymes are increasingly applied in the biotechnological and chemical industry, as well as in the medical diagnostics and therapie¹. They help to improve existing processes and make new applications possible. Therefore, there is an increasing demand of enzymes with new or improved properties. The improvement/optimization of enzymes by rational or computerized methods, however, has been mostly without success². Directed evolution of proteins, on the contrary, has been quite successful in optimizing properties of enzymes³. Directed evolution consists of two steps. Firstly, a gene library is created by varying one or more DNA sequences encoding the according enzyme and secondly, using selection or screening methods, those variants of the genes are isolated that encode enzymes variants with the desired, optimized properties. The advantage of this approach is that it does not require any information on the structure of the enzyme in question, its dynamics or its interactions with different substrates. Therefore, methods of directed evolution are increasingly accepted and applied by many biotech companies worldwide^{1,4}.

Success and failure of directed evolution above all depend on the quality of the libraries and the efficiency of the selection or screening method. The quality of a library is determined by the available sequence variation and depends mostly on the method used to create the sequence variation. Nowadays mainly two approaches are used to create sequence variation: in-vitro DNA-recombination⁵⁻⁷ and random mutagenesis⁸⁻¹⁰.

In-vitro DNA-recombination is based on a small repertoire of DNA sequences that are similar but not identical to each other. The differences (mutations) between these sequences can originate from natural evolution (family shuffling)¹¹ or could have been introduced by a combination of random mutagenesis and selection⁵. They therefore represent a pre-selection and in general improve or at least are neutral to the properties of the enzyme encoded by the DNA sequence.

In the in-vitro DNA-recombination a repertoire of DNA sequences is produced, the variants of which contain new combinations of the mutations that were present in the original repertoire. The advantage of this approach is that a high number of positions in a gene are varied at once and that mostly advantageous mutations are recombined. The disadvantage of this approach is that variation is confined to sequence positions that show variation in the original repertoire and additionally to the types of mutations present at these positions.

Random mutagenesis, on the contrary, provides access to completely new mutations. It has the advantage that all positions of a gene can contain every possible variation. Random mutagenesis, therefore, is not limited to mutations that are already present somewhere. Nowadays there are two techniques that are almost exclusively used for introducing random mutations into genes: error prone PCR⁸ and site specific (cassette-) mutagenesis using degenerated oligonucleotides⁹.

Error Prone PCR

The by far most dominant method of the site-nonspecific random mutagenesis is to use error prone PCR. Here the gene to be mutated is amplified by PCR using a thermostable polymerase (usually Taq-polymerase) that introduces wrong nucleotides in a rate that depends on the conditions of the PCR einbaut¹². A common variant of this method applies Manganese(II)-Ions⁸ or nucleotide analoga¹³ to adjust the error rate to suitable levels. Error prone PCR is inexpensive and simple, but it has the following drastic disadvantages:

1.) Due to the construction of the genetic code—three nucleotides encode one amino acid, each amino acid is encoded by up to six different codons—a single nucleotide exchange can only lead to 9 new codons (three new nucleotides on three positions in the codons) which on average give rise to only 6 different amino acids. Many amino acid exchanges can only be achieved when two or even all three nucleotide in a codon are exchanged. For example, a codon for Isoleucine (ATT, ATA, ATC) can only be gained by two exchanges when starting from the codon CAA (Glutamine) or even three exchanges when starting from the codon CAG (also Glutamine). There are examples from applications of mutagenesis for enzyme improvement where the exchange of all three nucleotides of a codon was indeed necessary to achieve the required properties of the enzyme^{14, 15}.

A repertoire of variants of a protein that contains all single amino acid mutations (each amino acid is represented on each position of the protein) has to be very large when it is produced by single nucleotide exchanges (e.g. error prone PCR). An average protein of 300 amino acid length has exactly 300×19=5700 variants that differ to each other by exactly one amino acid. A repertoire of variants of the same protein, which was produced by on average three nucleotide exchanges contains (900×3)ˆ3=2×10¹⁰ different variants. Repertoires of such sizes can only be handled by few selection methods¹⁶. Error prone PCR, therefore, can not be used to obtain complete high quality repertoires.

2.) Error prone PCR does not exchange all nucleotides alike. Transversions and transitions occur with different rates¹⁷ so that some mutations occur more frequently than others do. This effect leads to a further diminishing of the effective size of a repertoire produced by error prone PCR.

3.) The high redundancy of the codons—several codons encode the same amino acid—leads to the phenomenon that on average 23% of all nucleotide exchanges result in synonymous mutations in which the mutated codon encodes the same amino acid as the original one. Such mutations might lead to desired changes in the expression rate of proteins¹⁸, important intrinsic properties of the a protein, like for example the enzymatic activity or stability, however, are unchanged. The effect of this phenomenon is that the effective size of a repertoire is diminished.

4.) On average 4% of all nucleotide exchanges introduce new stop codons. By choosing a high mutation rate to achieve a maximum number of amino acid exchanges, many stop codons can be introduced which leads to shortened gene products. A repertoire produced with an average mutation rate of three exchanges per gene (in theory necessary to place at least all amino acids on one position) has already more than 10% prematurely terminated gene products [1−(1−0.04)ˆ3]=0.115.

5.) The introduction of mutations is mostly a stochastic process in which the number of mutations per gene in each single variant in one repertoire follows a Poisson distribution. Depending on the average mutation rate a significant part of the repertoire can have no mutation at all while others contain a very high number of mutations. The result is a further diminishment of the effective size of the repertoire.

6.) The introduction of non-natural amino acids in the biosynthesis of proteins can be achieved by using modified components of the complex biochemical apparatus for protein translation together with new codons that consist of four instead of three bases¹⁹. To generate protein variants that contain such non-natural amino acids at random positions complete codons, meaning three consecutive nucleotides have to be exchanged against four nucleotides. This is effectively impossible.

Site Directed Random Mutagenesis

An alternative for error prone PCR is to exchange several nucleotides at once at several defined positions in a gene by using degenerated oligonucleotides and PCR. When all three nucleotides of one codon are varied freely, any natural amino acid can be encoded at the position of this codon within the repertoire²⁰. However, a repertoire obtained such a way is distributed rather unevenly. For example, Arginine and Serine are represented 6 times as often as Methionine or Tryptophane. Additionally, the introduction of stop codons can be problematic. The degree of degeneracy of the oligonucleotides can be adjusted by using combinations or mixtures of certain nucleotides at different positions and such that some of these problems are circumvented. So, for example, DNA sequence repertoires can be obtained that encode only a specific part of all amino acids or that encode all amino acids with the same frequency^{21,22}.

In theory a better alternative for nucleotide mixtures in the chemical synthesis of DNA strands is the usage of nucleotide triplets, entire codons so to say, instead of single nucleotides²³⁻²⁷. Unfortunately the different tri-nucleotides all show a different efficiency of the chemical coupling during DNA synthesis, leading again to unevenly distributed repertoires²⁷. And even in the case that all these problems can be overcome, site directed random mutagenesis, as its name implies, is always limited to pre-selected, defined sites in a gene.

All presented limitations of the currently applied methodologies for random mutagenesis clearly show, how important and advantageous it was, to have a method that could:
1.) exchange complete codons (independent on the number of nucleotides within a codon) instead of single nucleotides randomly along the entire strand of DNA, and
2.) introduce a defined number of mutations (i.e. codon mutations) per each strand of DNA.

The invention presented here exactly describes such a method. Repertoires of gene variants prepared by the invented method have a better quality than repertoires that were prepared by conventional methods of error prone PCR. They contain a higher number of different variants while having the same number of repertoire members. When coupled to a selection or screening variants that encode proteins with new and desired properties can be isolated more efficiently from these repertoires. These proteins, for example, can be applied as enzymes in the biotechnological industry, in medical diagnostics or therapy.

The above features and many other advantages of the invention will be become better understood by reference to the following detailed description when taking in conjunction with the accompanying drawings.

### DESCRIPTION OF FIGURES

FIG. 1:

(A) Schematic description of the insertion of a Donor-strand of DNA (dark box) into random positions of a gene (open box), which is in a circular form (e.g. Plasmid). The Donor-strand can carry the gene of a reporter protein.

(B) The Donor-strand is removed in such a way that the original gene is recovered with the exception that one codon (shown as examples are CAG and CTG) is replaced by a different one (shown as examples are ATT and TAC.

The numbers exemplary represent the numbering of amino acids.

FIG. 2:

Detailed schematic description of the separate essential steps for the introduction of codon mutations into a gene. For detailed explanation the description of the invention below is referred. The dashed line symbolizes the circularity of the gene; it could show, for example, a plasmid. The numbers correspond to an exemplary numbering of the amino acids. The arrows above the genes mark restriction sites (not recognition sites) of restriction enzymes R1, R2, R3 and R4.

FIG. 3:

Detailed course of the mutagenesis shown exemplary by the exchange of codon 86 from GFP (AGT, nt 256-258) against TGG (amino acid mutation S86W). (i) . . . Introduction of a double strand break, (ii) . . . Insertion of the Donor-strand, (iii) . . . Restriction digestion with BseRI, (iv) . . . Creating blunt DNA ends, (v) . . . Religation.

On top of the DNA sequences nucleotide numbering is shown, below the DNA sequences the codon (amino acid) numbering is shown. The recognition sites for BseRI are in bold letters; the actual cutting sites are marked with dashed lines.

FIG. 4:

Agarose gel electrophoreses of PCR products to analyze the position of the placement of the Donor-strand with the GFP gene. The ladder on the side shows the size of the fragments in bp.

### DEFINITIONS

Before the detailed description of the invention several terms are defined:

DNA (deoxyribonucleic acid), polynucleotide, nucleotide sequence or oligonucleotide means any chain or sequence of the chemical building blocks Adenine (A), Cytosine (C), Guanine (G) and Thymine (T) (nucleotide bases). Nucleotides and oligonucleotide are degenerate, when they can have more than one nucleotide base at on or more positions. DNA can consist of one strand of nucleotide bases (single strand) or two complementary strands (double strand), which form a double helical structure. The term single strand break refers to breakage of one strand in double strand DNA after which the double strand is maintained. The term double strand break refers to breakage of both strands of double strand DNA after which two new DNA ends arise. Blunt DNA ends means that the ends of both strands in double strand DNA are equally long, overhanging or sticky DNA ends means that one strand is longer than the other. A blunt-end-ligation means that two double strands with blunt DNA ends are covalently attached to each other.

The terms library, repertoire or ensemble, as they are used here are identical and mean a collection of polynucleotides or polypeptides. A gene- or DNA-library or a repertoire of gene or DNA-sequences is a collection of polynucleotides or DNA-sequences, that are derivatives of on ore more polynucleotides or DNA-sequences and that are in most parts identical to each other.

Each single polynucleotide or each single DNA-sequence of a library is referred to as a member of the library. Several members of a library that are identical to each other are referred to as one gene variant or as one variant of the library. The effective size of a library or of a repertoire is a measure of the number of different variants within this library. A large library has many members, but when it had only a small effective size it had only few different variants.

Enzymes or terms of molecular biology as restriction enzyme, restriction enzyme of type IIs, DNase I, Nuclease, Exonuclease, DNA-Ligase, DNA-Polymerase, Transposase, Transposon, vector and plasmid are defined in their function as they are described in state of the art literature about molecular biology^{28, 29}.

### DETAILED DESCRIPTION OF THE INVENTION

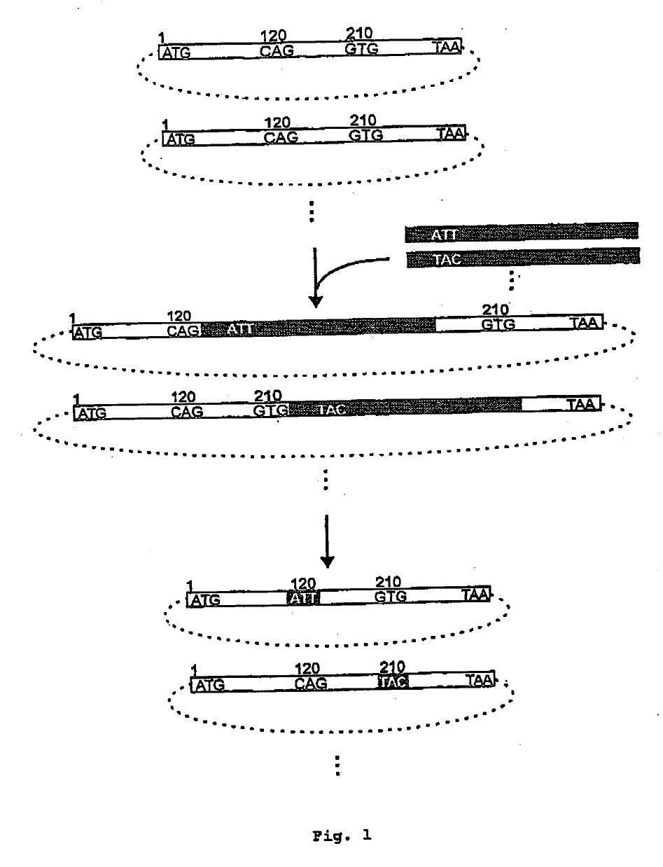

The invention consists of (also refer to FIG. 1):

A.) The insertion of a piece of DNA into a gene at different, randomly positioned sites.

B.) The directed removal of this piece of DNA and of a defined number of adjacent nucleotides of said gene, while instead at this position a defined number of nucleotides from said inserted piece of DNA is remaining.

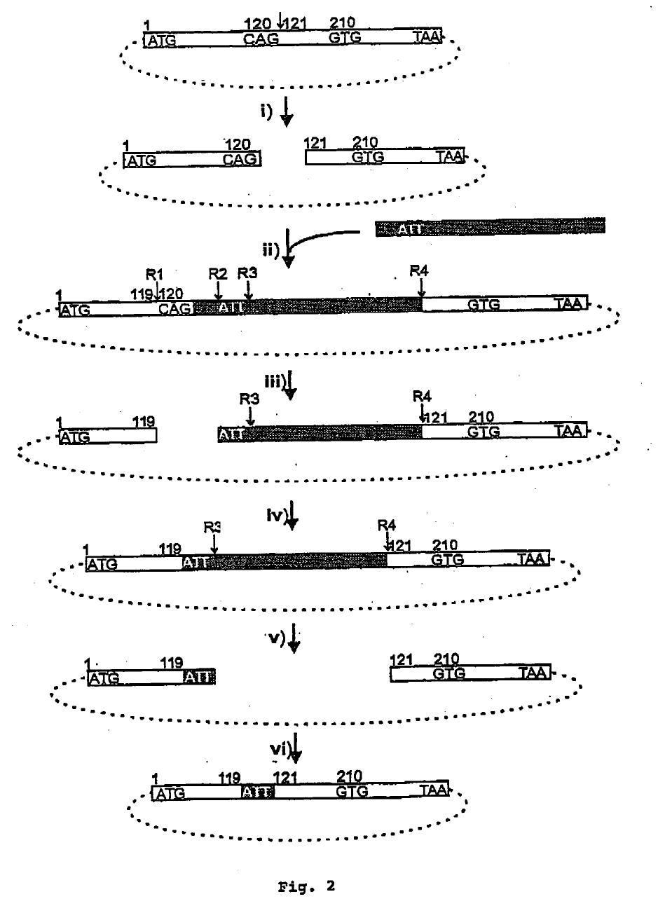

In detail the invention is marked by the following steps (also refer to FIG. 2):

1.) Into molecules of a gene or of a DNA sequence, preferably as part of a vector or any other circular form, exactly one double strand breakage per molecule is performed by mans of molecular biological methods (FIG. 2,i). This can, for example, be achieved by treating the DNA with a) an enzyme that site-nonspecifically introduces single strand breaks (e.g. DNase I) and a single strand specific nuclease (e.g. S1 nuclease), b) an enzyme that site-specifically introduces single strand breaks, a 5′-3′ exonuclease, DNA-polymerase and single strand specific nuclease (e.g. S1 nuclease), c) an enzyme that site-nonspecifically introduces double strand breaks (e.g. modified variants of restriction enzymes that have lost their sequence specificity), or d) a transposon and a transposase that does not have a sequence specificity. It is preferred that an ensemble of gene variants is produced in which the double strand breaks are located at different positions and (apart from case d) in which the double strand breaks lead to blunt DNA ends. In case d) the double strand breakage is achieved under simultaneous incorporation of a DNA strand (transposon) and possibly the doubling of several nucleotides from the gene.

2.) Into the gene variants of this ensemble a DNA strand (Donor-strand) is incorporated by blunt-end-ligation (FIG. 2,ii). In the case of the utilization of a transposase (1d) the Donor-strand, as a transposon, has been incorporated already during the previous step. It is preferred that the Donor-strand encodes a genetically selectable marker, e.g. a resistance against an antibiotic. It is further preferred that the expression of this marker is dependent on that the inserted Donor-strand is incorporated into the correct reading frame of the gene. Preferably, the Donor-strand contains recognition sites for restriction enzymes of type IIs. Such obtained DNA constructs can be completely or partially amplified by PCR^{30, 31} and/or can be amplified in and isolated from microorganisms after having transformed them. It is preferred that the growth of the microorganisms is performed in culture media containing antibiotics against which the microorganisms are resistant due to the gene product that is encoded on the Donor-strand.

3.) By restriction digestion with said restriction enzymes of type IIs the Donor-strands are mostly removed from the amplified gene variants. The DNA ends are made blunt by treatment with a DNA Polymerase or with a single strand specific nuclease (FIG. 2,iii). It is preferred that the positions of the recognition sites of said restriction enzymes of type IIs are chosen such that in addition to the removal of most of the Donor-strand a defined number of n nucleotides is removed from the original gene and that at their position a defined number of m nucleotides from the original Donor-strand is remaining. It is preferred that these remaining m nucleotides are degenerate, meaning that in the gene variants of the ensemble different nucleotide compositions are remaining. It is preferred that exactly three nucleotides are replaced (n=3, m=3).

In case that the variability of the nucleotide composition in the close proximity (10 to 40 base pairs) of the ends of the Donor-strand is restricted, e.g. by conserved sequences of a transposon, it is preferred that the Donor-strand contains several recognition sites for restriction enzymes of type IIs. These are preferably positioned in a way that the Donor-strand is removed from the gene bit by bit in several cycles of a) restriction digestion with one or two of said enzymes, b) when necessary, treatment to create blunt DNA ends and c) followed by the fusion of the DNA ends by intramolecular ligation, until the entire Donor-strand apart from m nucleotides is removed together with a defined number n nucleotides from the original gene (FIG. 2,iv and v). These remaining m nucleotides preferably are degenerate, it is preferred that exactly 3 nucleotides are replaced (n=3, m=3).

4.) By intramolecular blunt-end-ligation the DNA-ends of the variants of the ensembles are closed and complete, continuous genes are obtained (FIG. 2,vi). The genes can be subjected to a further round of introduction of mutations. Alternatively the genes can be expressed in vivo after transformation of an expression host or in vitro by using an in vitro—translation system to yield the protein variants that are encoded by the genes.

The here disclosed method is so far the only method for mutagenesis that allows the random exchange of several adjacent nucleotides in a gene. So far several methods of molecular biology have been published that are based on random double strand breaks of DNA strands or on the insertion of DNA sequences, including transposons at random positions into DNA strands. These methods, however, are limited to the experiments to find new termini for proteins³², to randomly delete parts of protein sequences or insert additional sequences into proteins³³. They have not been applied and, by themselves, they are not even applicable to exchange nucleotides at random positions in DNA sequences in such a way that single amino acids in the accordingly encoded proteins are exchanged to produce a repertoire of genes whose products are distinguished to each other in the type of a defined number of amino acids.

What is Predicted:

It is predicted that the fraction of any theoretically possible mixture of nucleotides within the degenerate part of the Donor DNA can be accurately adjusted, e.g. in such a way that in an area of 3 degenerate nucleotides each amino acid is represented by exactly one codon. It is predicted that the disclosed method allows incorporating mutations not only into the entire lengths of a gene but also into limited parts of genes. For example, after incorporation of the Donor-strands these parts can be amplified by PCR using flanking primers, the amplified products can be fused into the complete gene by the use of GenSOEing³⁴ and the such modified gene can then be further subjected to the described protocol. It is further predicted that during the stepwise removal of the Donor-strands, required restriction sites of type IIs are only created in the process, e.g. by restriction and religation. It is predicted that Donor-strands that are incorporated into genes as transposons by the action of a transposase can be modified with mutations within the transposase recognition sequence that necessary for the transposition, such that new recognition sites for restriction enzymes of type IIs are created within the transposase recognition sequence. It is predicted that there are other techniques that can be applied to introduce double strand breaks into DNA than the ones exemplary indicated in the description of the invention. It is predicted that by applying immobilization techniques genes with incorporated Donor-strands can be physically separated from genes that do not contain Donor-strands or that by applying immobilization techniques Donor-strands that are incorporated into genes can be physically separated from Donor-strands not incorporated into genes.

### EXAMPLE

The possibilities and the approach of the invention will become even clearer in the following example. The example of practicing the invention is understood to be exemplary only, and do not limit the scope of the invention or the appended claims. A person of ordinary skill in the art will appreciate that the invention can be practiced in many forms according to the claims and disclosure here.

### Example 1

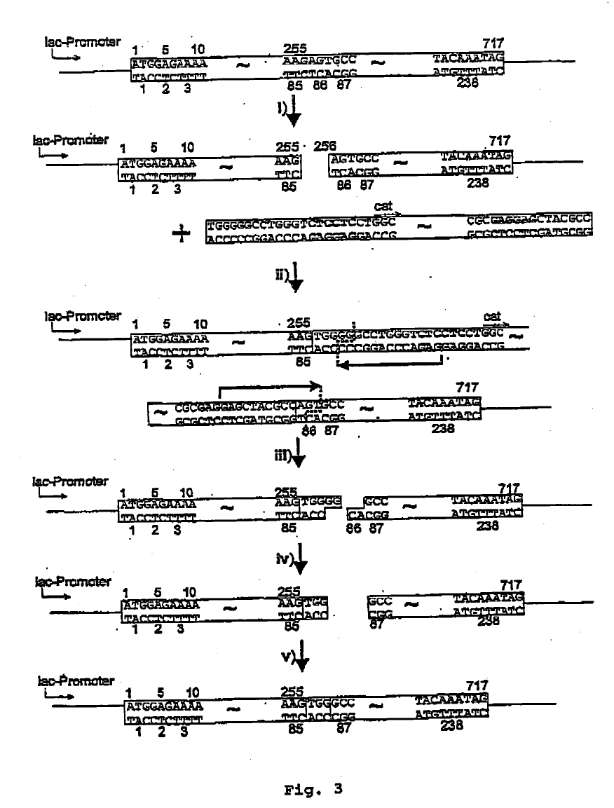

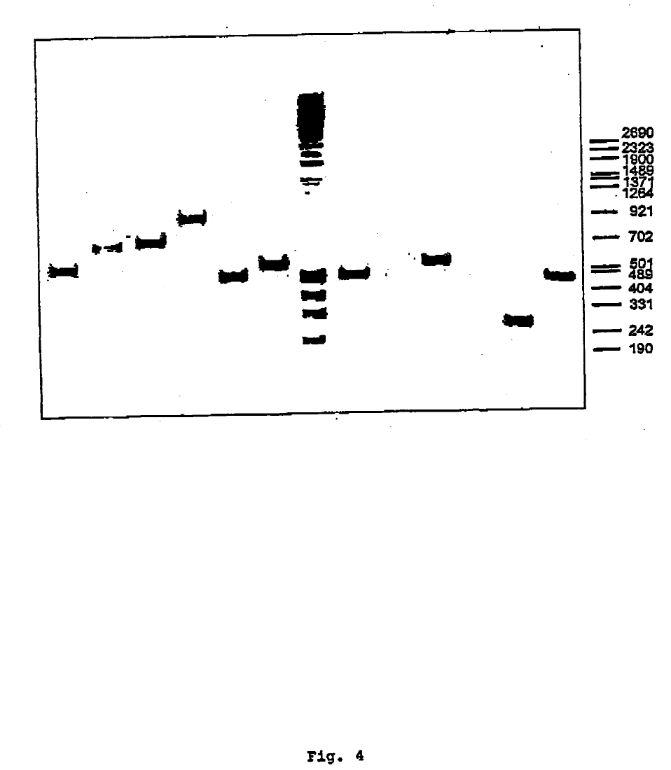

### Introduction of Codon Mutations into the Gene of the Green Fluorescent Protein (GFP) (Also Refer to FIG. 3 and 4)

1.) Introduction of Randomly Positioned Double Strand Breaks in the Plasmid pGFP

The plasmid pGFP (Clontech, Palo Alto, USA) contains the GFP-gene under the control of the lac-promoter. For the amplification in E. coli the plasmid contains the gene for the resistance against ampicillin. E. coli XL1-Blue cells were transformed with pGFP and from 200 ml of a culture of the transformed cells 300 μg pGFP DNA were prepared (Maxikit, Quiagen, Hilden, Germany).

In 200 μl 33 mM Tris/HCl, pH 7,5, 10 mM MgCl₂ and 50 μg/ml BSA 40 μg pGFP were incubated with 0.01 mu DNase I (Roche Diagnostik, Penzberg, Germany) for 5 min at 28° C. The reaction was stopped by addition of 20 mM EDTA (final conc.) and cooling on ice. The analysis of the reaction by agarose gel electrophoresis revealed that approx. 40% of pGFP had been converted into the open-circular form. This open circular DNA was isolated using preparative agarose gel electrophoresis. In 100 μl 7.4× S1 Buffer (MBI Fermentas, St. Leon Roth, Germany) 5 μg of the open-circular form were incubated with 100 u S1 Nuclease (1 μl, MBI Fermentas, St. Leon Roth, Germany) for 2 h at 16° C. after which the reaction was stopped with 10 μl S1-Stop solution. The analysis of the DNA by agarose gel electrophoresis revealed that approx. 50% of the open circular DNA was linearised. This linearised DNA was isolated by preparative agarose gel electrophoresis.

2.) Preparation of the DNA Strand to be Inserted (Donor-Strand)

The gene of chloramphenicol acetyltransferase (CAT) was amplified by PCR with the primers NNS GGG CCT GGG TCT CCT CCT GGC GAG AAA AAA ATC ACT GGA TAT ACC (SEQ. ID NO: 1) and GGC GTA GCT CCT CGC GTT TAA GGG (SEQ. ID NO: 2) and the Plasmid pACYC184 (NEB, Beverly, Mass., USA) as template. The PCR was performed following standard protocols (NEB, Beverly, Mass., USA), 30 cycles were performed applying an annealing temperature of 55° C. and an extension time of 45 sec. Vent-Polymerase was used (NEB, Beverly, Mass., USA). The PCR product was precipitated with EtOH and resuspended in a small volume TE to give a concentration of 150 ng/μl.

3.) Insertion of Donor-Strand into the Plasmid and Transformation

In 50 μl ligase buffer (Gibco BRL, Eggenstein, Germany) (final volume) 10 μl linearised plasmid (approx. 300 ng, refer to 1.) and 14 μl PCR product (approx. 2 μg, refer to 2.) were incubated with 5 u T4-DNA ligase (Gibco BRL, Eggenstein) for 20 h at 16° C. Subsequently the ligation mix was desalted by microdialysis and used to transform XL1-Blue cells by electroporation. Transformed cells were plated on dYT-Agar including 100 μg/ml Ampicillin, 8 μg/ml Chloramphenicol and 1 mM IPTG. Growth of transformed bacteria was basically limited to cells transformed with plasmids that contained the PCR fragment under the control of the lac-promoter in the correct reading frame after a start codon. Approx. 10000 transformants were obtained.

4.) Analysis of Library

95 colonies were analyzed by colony-PCR using the primers CCA TGA TTA CGC CAA GCT TGC (SEQ. ID NO: 3) (binds to the 5′-end of the GFP-gene and GTG CTT ATT TTT CTT TAC GGT C (SEQ. ID NO: 4) (binds within CAT-gene) for whether an insertion of the PCR-product into the plasmid had occurred within or outside the GFP gene and whether the insertion into the GFP gene had occurred in the correct direction of translation and at positions randomly distributed. From approx. 80% of the transformants a fragment between ca. 270 to ca. 1000 bp in length could be amplified (see FIG. 4a as an example. For all those variants the insertion of the PCR-product had occurred within the gene sequence of GFP in a way that the direction of translation of the gene for chloramphenicol acetyltransferase lies in the same as for the gene for GFP.

5.) Donor-Removal

All colonies of the transformed bacteria were collected from the agar plates, pooled and plasmid DNA was prepared (Mini kit, Quiagen, Hilden, Germany). 2 μg of the plasmid DNA, which represents a repertoire of pGFP with randomly inserted PCR products, was completely digested with BseRI (NEB, Beverly, Mass., USA). This restriction enzyme of Type IIs cuts outside its recognition site CTCCTC (FIG. 3,iii). The products of the restriction digestion were treated with Klenow fragment, subsequently separated by agarose gel electrophoresis and the DNA band that had a length of approx. 3.4 kb was isolated from the agarose (QiaexII, Qiagen, Hilden, Germany). Ca. 40 ng of the DNA were incubated in 50 μl ligation buffer with 1 u T4-DNA-Ligase for 20 h at 16° C. Subsequently the ligation mix was desalted by microdialysis and 5 μl were used to transform XL1-Blue cells by electroporation. Transformed cells were plated on dYT agar including 100 μg/ml Ampicillin.

6.) Second Analysis of Library

The transformants should contain the desired library of codon-mutated variants of GFP. For the analysis of this library 5 transformants were randomly selected and the entire sequence of GFP was determined to establish type and location of the mutation. The following mutations were found: S86W (AGT→TGG), G51H (GGA→CAC), N164R (AAC→CGC) and V219N (GTC-→AAG). One mutation was not in the correct reading frame and lead to the double mutation Q204L, S205A (CAATCT→CTCGCT).

6.) Phenotypic Analysis

The library of variants of GFP can be examined for variants that show a desired phenotypic change compared to wildtype GFP (e.g. increased expression of GFP, shift of excitation or emission wavelength, etc.). Desired variants can then be isolated and applied according to their properties.

### LIST OF REFERENCES

1. Rubingh, D. N., Protein engineering from a bioindustrial point of view. Curr. Opin. Biotechnol., 1997. 8(4): p. 417-22.
2. Chen, R., Enzyme engineering: rational redesign versus directed evolution. Trends Biotechnol., 2001. 19(1): p. 13-14.
3. Petrounia, I. P. and F. H. Arnold, Designed evolution of enzymes. Curr. Opin. Biotechnol., 2000. 11: p. 325-330.
4. Dordick, J. S., Y. L. Khmelnitsky, and M. V. Sergeeva, The evolution of biotransformation technologies. Curr. Opin. Microbiol., 1998. 1: p. 311-318.
5. Stemmer, W. P., DNA shuffling by random fragmentation and reassembly: in vitro recombination for molecular evolution. Proc. Natl. Acad. Sci. USA, 1994. 91(22): p. 10747-51.
6. Volkov, A. A., Z. Shao, and F. H. Arnold, Recombination and chimeragenesis by in vitro heteroduplex formation and in vivo repair. Nucleic Acids Res., 1999. 27(18): p. e18.
7. Zhao, H., et al., Molecular evolution by staggered extension process (StEP) in vitro recombination. Nat. Biotechnol., 1998. 16(3): p. 258-61.
8. Fromant, M., S. Blanquet, and P. Plateau, Direct random mutagenesis of gene-sized DNA fragments using polymerase chain reaction. Anal. Biochem., 1995. 224(1): p. 347-53.
9. Lahr, S. J., et al., Patterned library analysis: A method for the quantitative assessment of hypotheses concerning the determinants of protein structure. Proc. Nat. Acad. Sci. USA, 1999. 96(26): p. 14860-14865.
10. Greener, A., M. Callahan, and B. Jerpseth, An efficient random mutagenesis technique using an E. coli mutator strain. Mol. Biotechnol., 1997. 7(2): p. 189-95.
11. Crameri, A., et al., DNA shuffling of a family of genes from diverse species accelerates directed evolution. Nature, 1998. 391(6664): p. 288-91.
12. Tindall, K. R. and T. A. Kunkel, Fidelity of DNA synthesis by the Thermus aquaticus DNA polymerase. Biochemistry, 1988. 27(16): p. 6008-13.
13. Spee, J. H., W. M. de Vos, and O. P. Kuipers, Efficient random mutagenesis method with adjustable mutation frequency by use of PCR and dITP. Nucleic Acids Res., 1993. 21(3): p. 777-8.
14. Sawano, A. and A. Miyawaki, Directed evolution of green fluorescent protein by a new versatile PCR strategy for site-directed and semi-random mutagenesis. Nucleic Acids Res., 2000. 28(16): p. e78.
15. May, O., P. T. Nguyen, and F. H. Arnold, Inverting enantioselectivity by directed evolution of hydantoinase for improved production of L-methionine. Nat. Biotechnol., 2000. 18(3): p. 317-20.
16. Kuchner, O. and F. H. Arnold, Directed evolution of enzyme catalysts. Trends Biotechnol., 1997. 15(12): p. 523-30.
17. Cadwell, R. C. and G. F. Joyce, Mutagenic PCR. PCR Methods Appl., 1994. 3: p. S136-S139.
18. Komar, A. A., T. Lesnik, and C. Reiss, Synonymous codon substitutions affect ribosome traffic and protein folding during in vitro translation. FEBS Lett., 1999. 462(3): p. 387-91.
19. Martin, A. B. and P. G. Schultz, Opportunities at the interface of chemistry and biology. Trends Cell. Biol., 1999. 9(12): p. M24-8.
20. Oliphant, A. R., A. L. Nussbaum, and K. Struhl, Cloning of random-sequence oligodeoxynucleotides. Gene, 1986. 44(2-3): p. 177-83.
21. Balint, R. F. and J. W. Larrick, Antibody engineering by parsimonious mutagenesis. Gene, 1993. 137(1): p. 109-18.
22. Tomandl, D., A. Schober, and A. Schwienhorst, Optimizing doped libraries by using genetic algorithms. J. Comput. Aided Mol. Des., 1997. 11(1): p. 29-38.
23. Gaytan, P., et al., Combination of DMT-mononucleotide and Fmoc-trinucleotide phosphoramidites in oligonucleotide synthesis affords an automatable codon-level mutagenesis method. Chem. Biol., 1998. 5(9): p. 519-27.
24. Kayushin, A. L., et al., A convenient approach to the synthesis of trinucleotide phosphoramidites-synthons for the generation of oligonucleotide/peptide libraries. Nucleic Acids Res., 1996. 24(19): p. 3748-55.
25. Lyttle, M. H., et al., Mutagenesis using trinucleotide beta-cyanoethyl phosphoramidites. Biotechniques, 1995. 19(2): p. 274-81.
26. Ono, A., et al., The synthesis of blocked triplet-phosphoramidites and their use in mutagenesis. Nucleic Acids Res., 1995. 23(22): p. 4677-82.
27. Vimekas, B., et al., Trinucleotide phosphoramidites: ideal reagents for the synthesis of mixed oligonucleotides for random mutagenesis. Nucleic Acids Res., 1994. 22(25): p. 5600-7.
28. Smith, A. D., Oxford Dictionary of Biochemistry and Molecular Biology. 2 ed. 2000, Oxford: University Press.
29. McDonald, C. J., Enzymes in Molecular Biology. Essential Data Series, ed. D. Rickwood and B. H. Hames. 1996, Chichester: John Wiley & Sons.
30. U.S. Pat. No. 4,683,195
31. U.S. Pat. No. 4,683,202
32. Hennecke, J., P. Sebbel, and R. Glockshuber, Random circular permutation of DsbA reveals segments that are essential for protein folding and stability. J. Mol. Biol., 1999. 286(4): p. 1197-215.
33. Stone, J. C., et al., Identification of functional regions in the transforming protein of Fujinami sarcoma virus by in-phase insertion mutagenesis. Cell, 1984. 37(2): p. 549-58.
34. Horton, R. M. and L. R. Pease, Recombination and mutagenesis of DNA sequences using PCR, in Directed Mutagenesis—A Practical Approach, M. J. McPherson, Editor. 1991, IRL Press: Oxford. p. 217-247.

1-44. (canceled)

45. A method for producing sequence variation in DNA, which comprises the steps of:
a) incorporation of a transposon (DONOR) into said DNA (GEN) at different random positions and
b) specific removal of DONOR from GEN and specific removal of a defined number of adjacent nucleotides of GEN from GEN, such that at exactly the position of these removed nucleotides within the sequence of GEN a defined number of nucleotides remain that originate from DONOR and which can be completely degenerate.

46. A method according to claim 45, wherein step 1 (b) occurs by several cycles of the following steps (a) to (d), followed by the steps (e) to (g):
a) restriction digestion of GEN and at least parts of DONOR containing DNA using a restriction endonuclease of type IIs
b) by demand, treatment of the DNA-ends with enzymes that make the DNA-ends blunt and/or isolation of the GEN containing part of the restricted DNA
c) intramolecular ligation of the free DNA ends of the GEN containing part of the restricted DNA by which a circular strand of DNA is formed, which such receives a new recognition site for a restriction enzyme of type IIs and
d) by demand isolation and/or amplification of this circular DNA-strand
e) restriction digestion using at least one restriction enzyme of type IIs of the products obtained from the last cycle in step (c) or, when necessary of the amplified and/or isolated products from the last cycle in step (d)
f) treatment of the DNA-ends with enzymes that make DNA-ends blunt and/or isolation of the GEN containing part of the restricted DNA
g) intramolecular ligation of the free DNA-ends of the GEN containing part of the restricted DNA by which a circular DNA strand (cGEN') is formed, which has the original sequence of GEN with the exception of few nucleotides of GEN that were replaced by degenerate nucleotides from DONOR.

47. A repertoire of sequence variants of DNA that has been produced using a method according to claim 45.

48. A kit for creating sequence variation in DNA based on a method according to claim 45.

49. A method for producing sequence variation in DNA, comprising the steps:
a) introduction of a double strand breakage in said DNA (GEN),
b) ligation of a DNA Strand (DONOR) to both DNA-ends of GEN, formed by the double strand breakage of GEN, producing a ligation product (LP),
c) removal of the major part of DONOR from LP apart from few nucleotides that can be degenerate and removal of a small part of GEN from LP
d) intramolecular ligation of the free DNA-ends of the remaining part of LP such that a circular DNA strand (cGEN') is formed, which has the original sequence of GEN with the exception of few nucleotides of GEN that were replaced by degenerate nucleotides from DONOR.

50. A repertoire of sequence variants of DNA that has been produced using a method according to claim 49.

51. A kit for creating sequence variation in DNA based on a method according to claim 49.

52. A repertoire of sequence variants of DNA that has been produced using a method according to claim 2.

53. A kit for creating sequence variation in DNA based on a method according to claim 2.


SEQUENCE LISTING (12 sequences)

SEQ ID NO: 1 - DNA, 48 bp, Artificial Sequence
  synthetic oligonucleotide
nnsgggcctgggtctcctcctggcgagaaaaaaatcactggatatacc

SEQ ID NO: 2 - DNA, 24 bp, Artificial Sequence
  synthetic oligonucleotide
ggcgtagctcctcgcgtttaaggg

SEQ ID NO: 3 - DNA, 21 bp, Artificial Sequence
  synthetic oligonucleotide
ccatgattacgccaagcttgc

SEQ ID NO: 4 - DNA, 22 bp, Artificial Sequence
  synthetic oligonucleotide
gtgcttatttttctttacggtc

SEQ ID NO: 5 - DNA, 9 bp, Artificial Sequence
  synthetic sequence for GFP
atggagaaa

SEQ ID NO: 6 - DNA, 9 bp, Artificial Sequence
  synthetic sequence for GFP
aagagtgcc

SEQ ID NO: 7 - DNA, 9 bp, Artificial Sequence
  synthetic sequence for GFP
tacaaatag

SEQ ID NO: 8 - DNA, 24 bp, Artificial Sequence
  synthetic sequence
tgggggcctgggtctcctcctggc

SEQ ID NO: 9 - DNA, 16 bp, Artificial Sequence
  synthetic sequence
cgcgaggagctacgcc

SEQ ID NO: 10 - DNA, 27 bp, Artificial Sequence
  synthetic sequence
aagtgggggcctgggtctcctcctggc

SEQ ID NO: 11 - DNA, 22 bp, Artificial Sequence
  synthetic sequence
cgcgaggagctacgccagtgcc

SEQ ID NO: 12 - DNA, 9 bp, Artificial Sequence
  synthetic sequence
aagtgggcc